(19) United States
(12) Patent Application Publication
(10) Pub. No.: US 2025/0204755 A1
(43) Pub. Date: Jun. 26, 2025

(54) Articulation member for or in a deflection mechanism

(71) Applicant: KARL STORZ SE & Co. KG, Tuttlingen (DE)

(72) Inventors: Leonik Lander, 7 Dietingen (DE); Alexander Fuchs, Mühlheim (DE); Wolfgang Karl, Mühlheim (DE)

(73) Assignee: KARL STORZ SE & Co. KG, Tuttlingen (DE)

(21) Appl. No.: 18/987,455

(22) Filed: Dec. 19, 2024

(30) Foreign Application Priority Data
Dec. 20, 2023 (DE) 102023135953.3

(51) Int. Cl. A61B 1/00

(52) U.S. Cl. CPC A61B 1/00149; A61B 1/00098

(57) ABSTRACT

The present invention relates to an articulating member for or in a deflection mechanism at a distal end of a surgical instrument, in particular an endoscope, with a base body which has an axial through-opening along a longitudinal axis, so that a sub-portion of a working channel of the surgical instrument can be formed with the through-opening or can be passed through the through-opening, and a connecting portion arranged on at least one axial end portion of the base body, wherein the connecting portion is designed for radial engagement with a connecting portion of a further articulating member, so that a positive and articulated connection can be formed between two articulating members. The present invention further relates to a deflection mechanism and a method for producing a deflection mechanism.

### CROSS REFERENCE TO RELATED APPLICATIONS

This application claims priority to German Patent Application No. 10 2023 135 953.3 filed Dec. 20, 2023, and entitled “Artikulationsglied für eine oder in einer Auslenkmechanik,” which is incorporated herein by reference.

### TECHNICAL FIELD OF THE INVENTION

The present invention relates to an articulating member for a or in a deflection mechanism at a distal end of a surgical instrument. Furthermore, the invention relates to a deflection mechanism and a method for producing a deflection mechanism.

### BACKGROUND

A deflection mechanism for endoscopic instruments is used to angulate a tool or accessory attached to it, such as forceps, scissors, clamps, laser fiber or similar. The deflection mechanism therefore serves for tool alignment at a distal end of the surgical instrument, in particular an endoscopic instrument, such as an endoscope. Furthermore, an accessory for imaging, lighting and/or steering, in particular, by means of steering means such as pull wires or the like, can be guided using the deflection mechanism.

The deflection mechanism can also be referred to as an angulation unit or articulation unit, which has a through-opening with a working channel through which the accessory is passed. The accessory, such as forceps, scissors, clamps, laser fiber or similar, is usually pushed through the working channel of the endoscopic instrument. In addition to this working channel, a light and/or imaging unit may be permanently installed in the endoscopic instrument. In order to allow for angulating, the deflection mechanism is made of individual articulating members that are connected to one another in an articulated manner. The deflection or alignment of the deflection mechanism can be carried out with pull wires, which are also passed at least partially through the through-opening, in particular in separate openings.

Due to the large number of possible accessories as well as current- and light-conducting components, the through-opening should be as spacious as possible and free of obstructions. However, the connection of the individual articulating members may lead to cross-sectional constrictions inside, which reduce the cross section of the through-opening in portions. This is the case, for example, when the individual articulating members are connected to one another via connecting means, such as rivets, bolts or pins, in which case the connecting means protrude into the cross section of the through-opening and, in particular, represent angular obstructions. Furthermore, the connecting elements must be firmly connected to one of the articulating members so that it is securely mounted. This requires additional effort and associated time and costs for production.

To avoid this, a deflection mechanism is known from EP 1 604 607 A1, wherein hinge-shaped connections are formed between individual articulating members, wherein the individual articulating members are connected via a type of movable dovetail connection. A tenon is aligned in the longitudinal direction of the articulating member and engages in a recess of an adjacent articulating member. The recess has a semicircular shape, allowing rotation of the articulating members relative to one another. However, such a connection places high requirements on dimensional accuracy and precision, since the hinge joint is designed to be only as thick as the wall thickness of the articulating member, and the articulation mechanism itself already has a very small cross section. Furthermore, the maximum rotational movement of the two members is limited by the size of the semicircular recess. Particularly large angles can hardly be implemented because the remaining material thickness between the ends of the semicircular recess becomes very small as the circumference increases.

### BRIEF SUMMARY

Against this background, it is the object of the present invention to provide an improved articulating member as well as an improved deflection mechanism as well as the production thereof.

According to the invention, this object is achieved by an articulating member having the features of claim 1, by a deflection mechanism having the features of claim 11, and/or by a method having the features of claim 16.

Accordingly, the following are provided:
an articulating member for or in a deflection mechanism at a distal end of a surgical instrument, in particular an endoscope, with a base body which has an axial through-opening along a longitudinal axis, so that a sub-portion of a working channel of the surgical instrument can be formed with the through-opening or can be passed through the through-opening, and a connecting portion which is arranged on at least one axial end portion of the base body, wherein the connecting portion is designed for radial engagement with a connecting portion of a further articulating member, so that a positive and articulated connection can be formed between two articulating members.
a deflection mechanism for aligning, in particular for aligning tools and/or for aligning a light, flushing and/or imaging unit, at a distal end of a surgical instrument, in particular an endoscope, with a plurality of articulating members, wherein the articulating members are connected to one another in a positive and articulated manner such that an articulated working channel of the surgical instrument is formed through the through-openings or a flexible working channel can be passed through the through-opening.

A method for producing a deflection mechanism for aligning, in particular for aligning tools and/or for aligning a light, flushing and/or imaging unit, at a distal end of a surgical instrument, in particular an endoscope, comprising the steps: providing a plurality of articulating members, pushing, at least partially, adjacent articulating members into one another, wherein connecting portions of adjacent articulating members are displaced relative to one another until the connecting portions contact one another in a radial direction of the base body such that a positive and articulated connection of the articulating members is produced or can be produced.

The finding underlying the present invention is that a connection of the articulating members to one another without loose connecting means brings advantages and, in particular, allows for single use.

The idea underlying the present invention is to create an articulated connection of individual articulating members, which is possible solely from the geometry of the articulating members themselves, i.e., in particular, a geometry of axial end portions or edge regions of the articulating members.

The base body is designed, in particular, as a tubular or sleeve-shaped element which is open, in particular, at both end portions. This allows an axial through-opening to be formed along the longitudinal axis of the articulating member. The end portions preferably have a circular or elliptical cross section. Other cross sections are also conceivable.

A connecting portion is arranged on at least one end portion, which is designed for radial engagement with a connecting portion of another articulating member. For example, a large number of similar articulating members can be connected to one another to form an articulation mechanism or deflection mechanism.

A radial engagement is, in particular, a contact between two connecting portions of adjacent articulating members. Radial can mean any direction in a cross-sectional plane of the articulating member or the axial end portion. Thus, a connecting portion can be arranged at any position along a circumference of the axial end portion. In particular, a plurality of connecting portions can also be formed at different circumferential positions, which are each designed, in particular, for radial engagement with a connecting portion of the adjacent articulating member and, in particular, engage with the adjacent articulating member independently of one another.

A radial engagement also offers the advantage that a relatively large contact surface can be formed, which is independent of the cross section of the base body or the articulating member. This makes it possible to produce a stable and robust connection.

The connecting portions of adjacent articulating members are, in particular, designed to complement one another so that the two connecting portions can fully contact one another.

A positive and articulated connection between two articulating members allows for the articulating members to be pivoted or rotated relative to one another. An axis of rotation is formed, in particular, by the connecting portion, for example, transversely to the longitudinal axis.

The articulating member is designed for or in a deflection mechanism of a surgical instrument, wherein any accessory, such as, in particular, a working channel for a tool such as forceps, scissors, laser fiber, collecting basket or the like, can be deflected or guided by means of the deflection mechanism. Furthermore, an accessory for imaging, lighting and/or steering, in particular, by means of steering means such as pull wires as well as a flushing, suction and/or working channel or the like, can be guided by means of the deflection mechanism.

The plurality of accessories must be passed through the through-opening of the articulating member or the deflection mechanism. It is advantageous if the inner diameter of the through-opening is, on the one hand, continuously constant and, on the other hand, designed to be as free of “obstructions” as possible. In particular, in the case of very small diameters of the articulating members, such as diameters of less than or equal to 3 mm, stringent requirements are placed on the articulated connection between adjacent articulating members, so that these should be designed to save space. The proposed positive and articulated connection offers the advantage that it hardly, or, in particular, not at all, intervenes in the inner cross section of the base element. In particular, the protrusion of the connecting portion into the interior of the base element is so small that the working channel is not disturbed. The articulating member therefore offers advantages especially for particularly small diameters of the base body, in particular, for diameters in the millimeter range, preferably 3 mm or smaller.

The surgical instrument may be, in particular, an endoscope. The articulating member is also suitable for other surgical instruments or endoscopic instruments where miniaturization of the deflection mechanism is advantageous.

Advantageously, a rivet-free connection between articulating members can therefore be achieved, which can, in particular, be achieved solely from a positive connection between axial end portions. The articulating members can be easily assembled, wherein an articulated connection can be formed by resilient deformation of at least one end portion. The end portions of adjacent articulations can therefore lock or snap together, thereby forming an articulated connection.

Advantageous embodiments and developments are shown in the further dependent claims and in the description with reference to the figures in the drawing.

In a preferred embodiment, the connecting portion may be designed to be resilient, wherein the positive connection can be produced by a resilient deflection in the radial direction. Resilient means, in particular, that the connecting portion can be resiliently deflected in two opposite directions, so that the connecting portion resiliently deforms back to its original position when it is connected to the connecting portion of an adjacent articulating member. The articulating member is therefore preferably formed from a metal and may, in particular, be made by cutting it out of a tube.

In a preferred embodiment, the connecting portion may have at least one tab. A tab is understood to mean, in particular, a portion that protrudes from the axial end portion. The tab may, for example, define an articulation axis so that adjacent articulating members can be guided and deflected relative to one another.

In a preferred embodiment, at least one tab may protrude beyond the base body in a longitudinal direction of the articulating member and may be resiliently deflectable in a radial direction. Advantageously, the inner diameter of the base body is not affected, or, in particular, only slightly affected, by the tab. In particular, the tab is oriented such that it has a flat surface aligned in a tangential direction or in a circumferential direction. This creates a two-dimensional surface that can be used for radial engagement. An articulation axis may be formed within and transverse to this two-dimensional surface.

In particular, the tab can be brought into radial engagement with another tab of an adjacent articulating member. In a radial engagement, the two tabs can contact one another via respective tangential and/or circumferential surfaces of the relevant tab.

In a preferred embodiment, the at least one tab may be dome-shaped, wherein the positive and articulated connection can be formed by the tab contacting a tab of a further articulating member, which tab is complementary to the dome-shaped tab. In particular, the dome shape can be produced by a deforming process alone, without the need to remove or add material. The dome shape also provides full contact with an adjacent articulating member, which aligns itself after adjacent articulating members are inserted or snapped into place. Dome-shaped therefore means, in particular, that the tab has a curvature.

In a preferred embodiment, the dome-shaped tab may have a convex or concave dome in a radial direction of the base body. This allows a defined articulation axis to be implemented through a center point of the base body. In a convexly shaped dome, the dome is, in particular, shaped towards an outer surface of the base body, whereby the interior of the base body is not affected. In a concavely shaped dome, the dome is, in particular, aligned in the direction of an inner surface to a center point of the base body. As a result, the dome protrudes minimally into the through-opening, but due to the continuously shaped surface of the curved dome, it does not represent an angular obstruction on which accessories can get caught. Furthermore, in order to form an articulated connection, it is sufficient if the dome has only a small height up to the apex.

In a preferred embodiment, two tabs may be provided on at least one end portion, which are, in particular, formed opposite one another on a cross section of the end portion and/or mirrored to one another. Each of the tabs may be shaped as shown above. For example, a defined articulation axis can be implemented between the two tabs.

In a preferred embodiment, a connecting portion may be arranged at each of two end portions which are arranged opposite one another on the base body, so that the articulating member can be connected to another articulating member at each end portion. This allows a plurality of articulating members to be connected to one another, wherein the articulating members can all be constructed in the same way. For example, so-called end members can be used at the end portions of the deflection mechanism that have a connecting portion only at one end portion and are formed without a connecting portion at the other end portion.

In a preferred embodiment, the tab may be designed for articulated engagement with a connecting portion of the further articulating member on a predetermined articulation axis. In particular, the articulation axis may extend through the center point of the base body.

In a preferred embodiment, the through-opening may be designed for the passage and defined accommodation of steering means, in particular steering cables or steering wires. For this purpose, molded parts can be formed from the base body, which can serve as guide elements for the steering means. In particular, the molded parts may be at least partially connected to the base body and form a defined guide channel within the inner cross section of the base body.

In a preferred embodiment of the deflection mechanism, the connecting portions of adjacent articulating members may be arranged offset from one another in a plane, wherein, in a connected state, they are arranged one above the other and engage radially with one another. Offset in a plane means, in particular, that the connecting portions are arranged at different radial distances. For example, a connecting portion may be closer to a center point of the base body than a connecting portion of an adjacent articulating member. This allows the connecting portions to be pushed over one another when the articulating members are connected together. In particular, the connecting portions can lock or snap into one another, creating a positive and articulated connection.

An articulating member may have connecting portions at two opposite end portions, which are arranged offset from one another in a plane. This allows identical articulating members to be connected to one another to form a coherent deflection mechanism.

Irrespective of the embodiment, the tabs of adjacent articulating members are, in particular, aligned parallel to one another and/or formed complementarily to one another.

In a preferred embodiment of the deflection mechanism, the connecting portions may each have at least one tab, wherein at least one of the tabs can be resiliently deflected in a radial direction of the base body and, in a connected state, the tabs are locked together. In particular, the tab is resiliently deformable in two opposite directions in the radial direction, so that it is designed to be self-resetting.

In a preferred embodiment of the deflection mechanism, the tabs of adjacent articulating members may have a complementary shape, in particular, a complementary dome shape. This allows an articulation axis to be formed about which the two articulating members can be moved in any direction relative to one another in rotational manner. The angle of rotation is not limited by the tab or the dome shape, but solely by the shape of the base body, wherein side portions at the axial end portions of the base body determine how far the articulating members can be moved relative to one another before they touch.

In a preferred embodiment of the deflection mechanism, the deflection mechanism may be designed for single use. This is achieved, in particular, in that the individual articulating members of the deflection mechanism can be manufactured cost-effectively and without great expenditure of time. On the other hand, the connection of the articulating members to one another can be implemented with little expenditure of time by eliminating the need for additional connecting elements and can be implemented by simply snapping or locking the connecting portions. The deflection mechanism can be mounted on site without tools.

Single use offers several advantages. On the one hand, sterility is ensured from the outset and, in particular, cross contamination is prevented. Furthermore, there is no cost for cleaning and repairing damaged elements. Endoscopes, in particular, are very fragile and can quickly become damaged. A single-use deflection mechanism therefore offers the advantage that repairs are not necessary due to the cost-effective production. In contrast to reusable endoscopes, disposable endoscopes are therefore disposed of after use. Repair of damaged products can therefore be dispensed with. Furthermore, several surgical instruments are usually used in parallel. This means that a high level of availability must be provided in order to be able to use several deflection mechanisms simultaneously. If each deflection mechanism is cost-effective on its own, several deflection mechanisms can be kept in stock without representing a financial burden.

In a preferred embodiment of the method, the one connection of two adjacent articulating members can be formed by a deformation step following the pushing into one another, wherein contacting connecting regions pushed parallel over one another are deformed complementarily. For example, after being pushed together, two connecting portions, in particular, two tabs arranged parallel to one another, can be deformed into a dome shape in order to define an articulation axis. This allows, for example, to realize the accuracy and concentricity of the connection or the articulation axis without great effort.

In a preferred embodiment of the method, at least one connecting portion of each articulating member may be designed to be resilient, wherein the connecting portions lock together by a resilient deflection when pushed into one another. In such an embodiment, the connecting portions or tabs may be already formed into a dome shape before they are pushed together, so that the connecting portions or tabs center themselves, align themselves and connect to one another in a positive and articulated manner when pushed together using the convex or concave dome shape.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

### BRIEF DESCRIPTION OF THE DRAWINGS

The present invention is explained in greater detail below with reference to the exemplary embodiments shown in the schematic figures of the drawing. In the drawings:

FIG. 1 is an isomeric view of a deflection mechanism;

FIG. 2 is a detailed view from FIG. 1;

FIG. 3 is a further detailed view of the embodiment of FIG. 2;

FIG. 4 is a further detailed view of the embodiment of FIG. 2;

FIG. 5 shows an articulating member in an isometric view;

FIG. 6 is a sectional view through an articulating member;

FIG. 7 is a sectional view through the embodiment according to FIG. 1;

FIG. 8 is a sectional view through a further embodiment of a deflection mechanism;

FIG. 9 is a side view of the embodiment of FIG. 8;

FIG. 10 is a detailed view from FIG. 9;

FIG. 11 is a top view of the embodiment of FIG. 9;

FIG. 12 is a further view of the embodiment of FIG. 9; and

FIG. 13 is a sectional view through an articulating member for a deflection mechanism according to FIG. 9.

The accompanying figures of the drawing are intended to provide a further understanding of the embodiments of the invention. They illustrate embodiments and, in conjunction with the description, serve to explain principles and concepts of the invention. Other embodiments and many of the advantages mentioned are shown in the drawings. The elements in the drawings are not necessarily shown to scale.

In the figures of the drawing, identical, functionally equivalent, and identically acting elements, features, and components are each provided with the same reference signs, unless otherwise specified.

### DETAILED DESCRIPTION

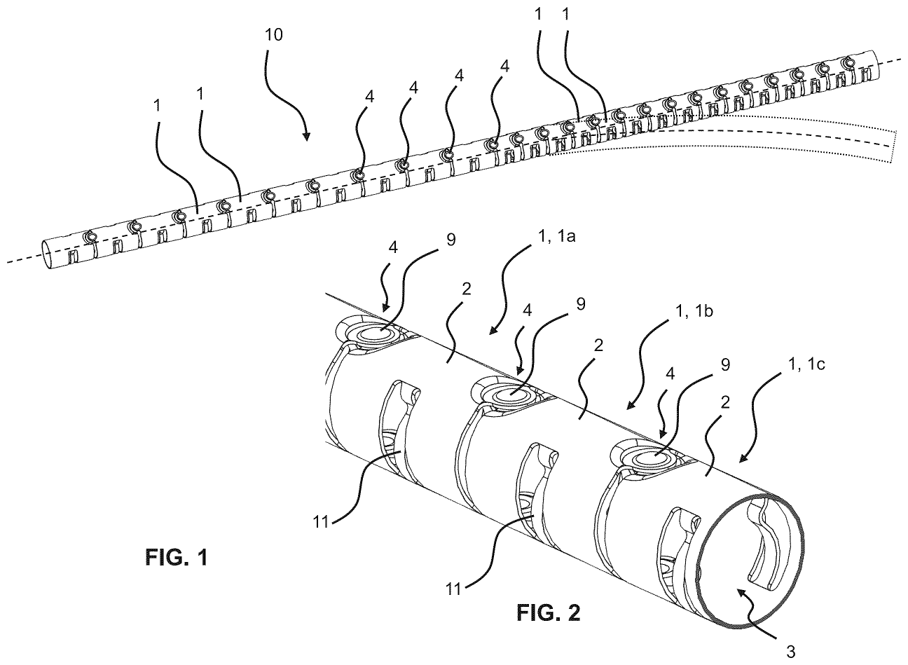

FIG. 1 shows an isometric view of a deflection mechanism 10. The deflection mechanism 10 is formed from a plurality of articulating members 1, which are connected to one another via connecting portions 4. As a result, the deflection mechanism 10 can be formed in an articulated manner so that it can be deflected from a longitudinal axis, as shown by way of example by the downwardly inclined and dashed view. The deflection mechanism 10 may have articulating members 1 of different lengths, whereby the radius of a deflection or a rotational movement can be affected.

FIG. 2 shows a detailed view from FIG. 1. Several articulating members 1a, 1b, 1c are shown, each having a base body 2. The base body 2 is, in particular, tubular or sleeve-shaped so that a through-opening 3 can be formed. Furthermore, molded parts 11 can be partially cut out of the base body 2 and deformed such that further guide channels 12 are formed within the through-opening 3. The guide channels 12 can be used, in particular, for passing a steering mechanism through.

The illustrated articulating members are shown in a connected state, wherein the connecting portions 4 of adjacent articulating members are connected to one another. In the embodiment shown, the connecting portions 4 each have a dome 9 which defines an articulation axis 13, which is shown in FIG. 6. The connecting portions 4 of adjacent articulating members 1 are, in particular, aligned parallel to one another and/or formed complementarily to one another.

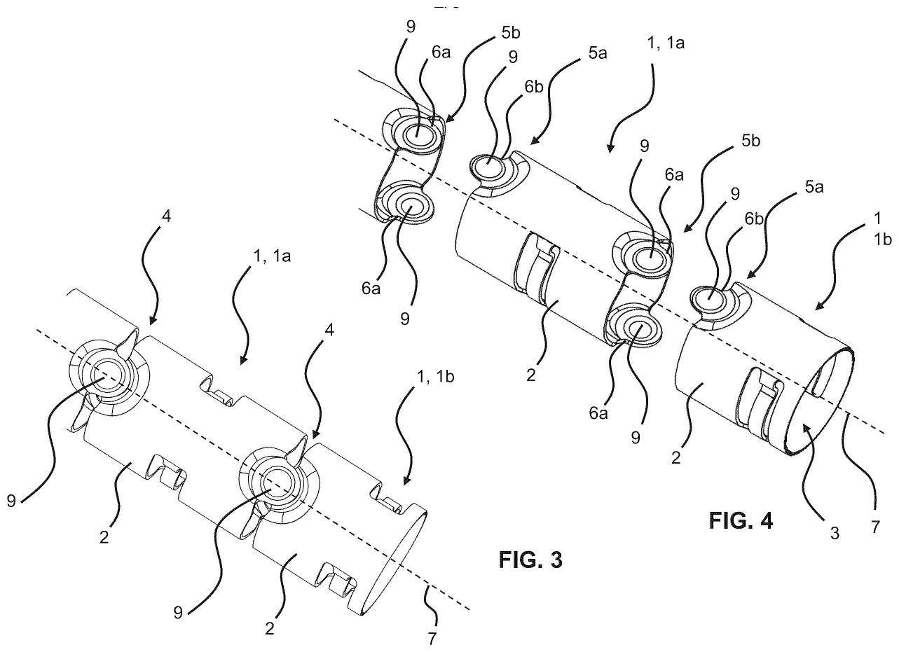

FIG. 3 shows a further detailed view of the embodiment according to FIG. 2. The articulating members 1a, 1b touch one another at the connecting portions 4 only in the region of the dome 9, wherein the remaining axial end portion of the articulating member 1a is arranged spaced apart from the axial end portion of the articulating member 1b. As a result, a deflection from the longitudinal axis 7 can be achieved, since the articulating members 1a, 1b can perform a rotational movement relative to one another. The dome 9 serves, in particular, to guide the rotational movement.

FIG. 4 shows a further detailed view of the embodiment according to FIG. 2. The articulating members 1 are shown prior to being pushed together. The articulating member 1a has a connecting portion at each of two oppositely arranged axial end portions 5a, 5b, which in this embodiment is designed as a tab 6a or tab 6b. In each tab 6a, 6b, a dome 9 is formed, which in this embodiment is convexly aligned in the radial direction. This can be seen, for example, from FIG. 6.

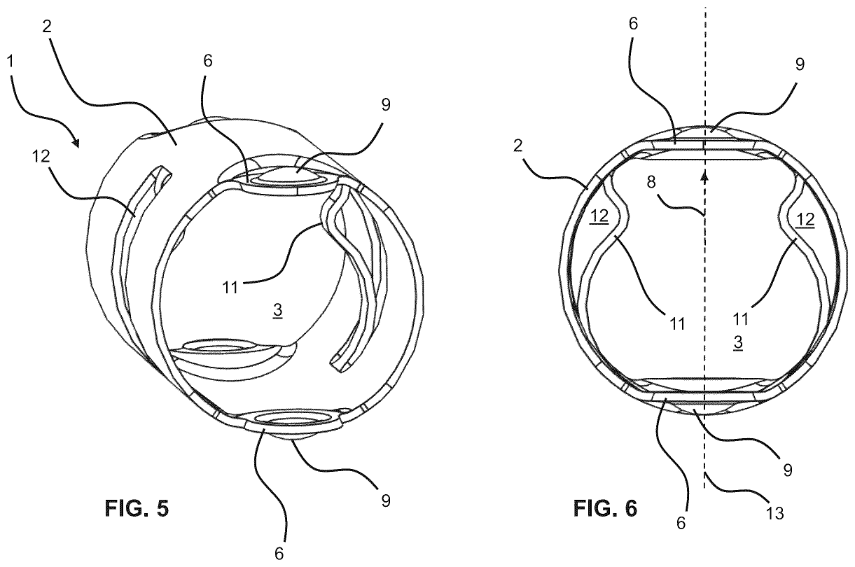

FIG. 5 shows an articulating member 1 in an isometric view and FIG. 6 shows a sectional view through an articulating member 1. Deviating from the embodiment shown, the tabs 6 may also be arranged on other circumferential portions of the base body 2 or the connecting portion 4. It is also conceivable that only one tab 6 is formed on an axial end portion. Since the dome 9 is curved in the direction of an outer surface of the base body 2, it does not protrude into the inner cross section of the through-opening 3. As a result, the inner cross section is not affected by the connecting portion 4. In particular, this makes it possible to achieve an almost constant and uniform inner cross section along the longitudinal axis 7.

In a further embodiment, molded parts 11 may be provided which are molded out of the base body 2 but are still connected to the base body 2. As a result, for example, guide channels 12 can be formed within the through-opening 3 as separate openings, which can serve, in particular, to guide a steering mechanism.

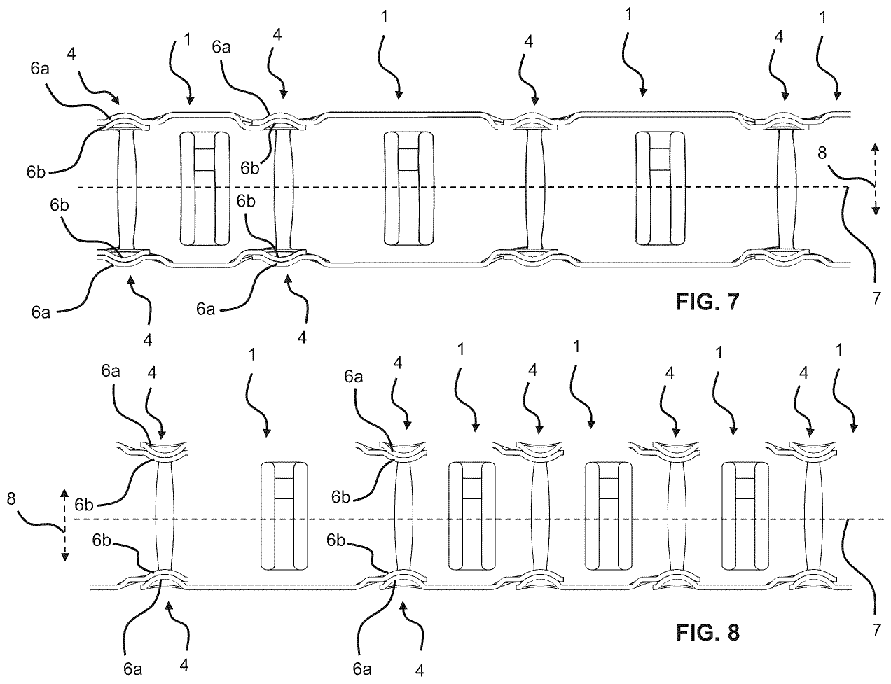

FIG. 7 shows a sectional view through the embodiment of FIG. 1. It can be seen how the two dome-shaped tabs 6a, 6b engage with one another.

The tabs 6a, 6b are, in particular, formed complementarily to one another, so that the two tabs 6a, 6b can fully contact one another.

FIG. 8 shows a sectional view through an embodiment of a deflection mechanism 10. In this embodiment, the tabs 6a, 6b are also dome-shaped, wherein the dome is concave with respect to the radial direction 8. The tabs 6a, 6b are therefore curved in the direction of a center point of the through-opening 3 so that they protrude minimally into the through-opening 3. However, due to the curved shape with a continuous surface, an obstruction for accessories is not created. Rather, the continuous dome shape can serve to guide the accessories when they are moved along the through-opening 3 and in the direction of the longitudinal axis 7. A side view of the embodiment of FIG. 8 is shown in FIG. 9.

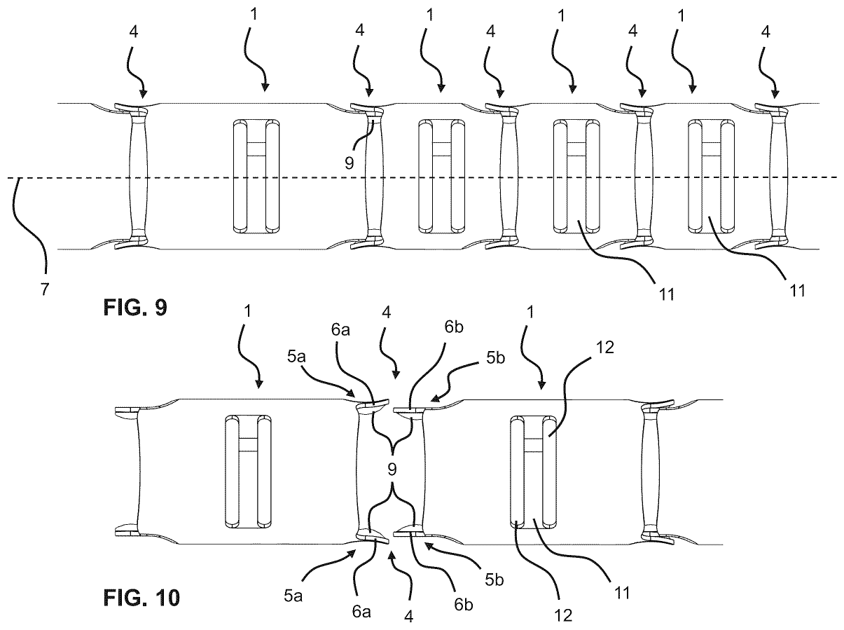

FIG. 10 shows a detailed view of the embodiment of FIG. 9, wherein the two articulating members 1 are shown prior to assembly, i.e., prior to being pushed into one another. It can be seen that the two domes 9 are formed complementarily by adjacent tabs 6a, 6b. It can also be seen that the tabs 6a provided with the reference signs of the articulating member 1 shown on the left are arranged in a plane offset from the tabs which are located on an opposite axial end portion (far left in the view) on the same articulating members 1. Such an embodiment makes it particularly advantageous for identically constructed articulating members 1 to be pushed into one another.

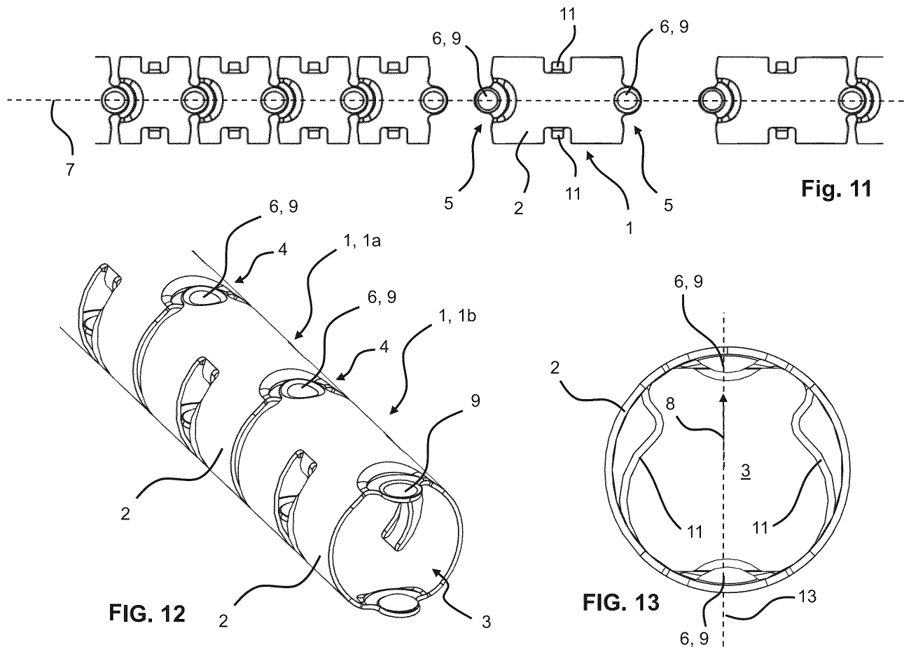

Further views of the embodiment according to FIG. 8 are shown in FIGS. 11 to 13.

Regardless of the embodiment, the articulating members 1 can be manufactured or cut out from a complete tube. This can be done, in particular, by laser cutting, plasma cutting, water jet cutting, punching or similar. The dome-shaped tabs can be formed through a deforming process. Since the tube consists, in particular, of a metal material, the tabs 6 or the connecting portions 4 are resiliently formed so that the articulating members 1 can be pushed into one another, wherein a connection can be established by resilient deformation.

The deflection mechanism 10 can be used, for example, for different surgical instruments, in particular, for endoscopes and also for catheters. The deflection mechanism 10 is designed, in particular, for small diameters of the articulating members 1. Thus, in particular, diameters of 2-5 mm, in particular 3 mm, preferably less than 3 mm, can be implemented.

The deflection mechanism 10 can, for example, also be mounted directly on site, since the assembly can be achieved by simply locking or snapping the individual articulating members 1 together.

The dome-shaped tabs can be formed either prior to or after the parts are pushed together. Advantageously, the dome-shaped tabs are formed prior to being pushed together so that they snap together as soon as they are pushed together. The connection geometries can deform resiliently in the radial direction, creating a positive and articulated connection. The positive connection offers the advantage that additional connecting elements, such as, in particular, rivets, bolts or the like, can be dispensed with.

The deflection mechanism 10 or the articulating members 1 are particularly suitable for single use, i.e., for a single-use application, since they can be manufactured cost-effectively and connected to one another in a time-saving manner.

A plurality of embodiments and combinations thereof represent aspects of the disclosed invention, including, but not limited to, the following variations.

An articulating member (1) for or in a deflection mechanism (10) at a distal end of a surgical instrument, in particular an endoscope, comprising: a base body (2) having an axial through-opening (3) along a longitudinal axis (7), so that a sub-portion of a working channel of the surgical instrument can be formed with the through-opening (3) or can be passed through the through-opening; and a connecting portion (4) arranged on at least one axial end portion (5) of the base body (2), wherein the connecting portion (4) is designed for radial engagement with a connecting portion (4) of a further articulating member (1), so that a positive and articulated connection can be formed between two articulating members (1).

The articulating member (1) described above, characterized in that the connecting portion (4) is designed to be resilient, wherein the positive connection can be established by a resilient deflection in the radial direction.

An articulating member described above, further characterized in that the connecting portion (4) has at least one tab (6).

An articulating member described above, further characterized in that the at least one tab (6) projects beyond the base body (2) in a longitudinal direction (7) of the articulating member (1) and is resiliently deflectable in a radial direction (8).

An articulating member described above, further characterized in that the at least one tab (6) is dome-shaped, wherein the positive and articulated connection can be formed by the tab (6) contacting a tab (6) of a further articulating member (1), which tab is complementary to the dome-shaped tab.

An articulating member described above, further characterized in that the dome-shaped tab (6) has a convex or concave dome (9) in a radial direction (8) of the base body (2).

An articulating member described above, further characterized in that two tabs (6) are provided on at least one end portion (5), which are, in particular, formed opposite one another on a cross section of the end portion (5) and/or mirrored to one another.

An articulating member described above, further characterized in that a connecting portion (4) is arranged at each of two end portions (5a, 5b) which are arranged opposite one another on the base body (2), so that the articulating member (1) can be connected to another articulating member (1) at each end portion (5a, 5b).

An articulating member described above, further characterized in that the tab (6) is designed for articulated engagement with a connecting portion (4) of the further articulating member (1) on a predetermined articulation axis (13).

An articulating member described above, further characterized in that the through-opening (3) is designed for the passage and defined accommodation of steering means, in particular, steering cables or steering wires.

A deflection mechanism (10) for aligning, in particular for aligning tools and/or for aligning a light, flushing and/or imaging unit, at a distal end of a surgical instrument, in particular an endoscope, comprising: a plurality of articulating members (1a, 1b) according to any of claims 1 to 10, wherein the articulating members (1a, 1b) are connected to one another in a positive and articulated manner such that an articulated working channel of the surgical instrument is formed through the through-openings or a flexible working channel can be passed through the through-opening.

The deflection mechanism (10) described above, further characterized in that connecting portions (4) of adjacent articulating members (1a, 1b) are arranged offset from one another in a plane, wherein, in a connected state, they are arranged one above the other and engage radially with one another.

A deflection mechanism (10) described above, further characterized in that the connecting portions (4) each have at least one tab (6), wherein at least one of the tabs (6) is resiliently deflectable in a radial direction (8) of the base body (2) and in a connected state the tabs (6) are locked together.

A deflection mechanism (10) described above, further characterized in that the tabs (6) of adjacent articulating members (1) have a complementary shape, in particular, a complementary dome shape.

A deflection mechanism (10) described above, further characterized in that the deflection mechanism (10) is designed for single use.

A method for producing a deflection mechanism (10) for aligning, in particular for aligning tools and/or for aligning a light, flushing and/or imaging unit, at a distal end of a surgical instrument, in particular an endoscope, comprising the steps of: providing a plurality of articulating members (1a, 1b) described above, pushing, at least partially, adjacent articulating members (1a, 1b) into one another, wherein connecting portions (4) of adjacent articulating members (1a, 1b) are displaced relative to one another until the connecting portions (4) contact one another in a radial direction (8) of the base body (2) such that a positive and articulated connection of the articulating members (1a, 1b) is produced or can be produced.

The method for producing a deflection mechanism (10) for aligning described above, characterized in that a connection of two adjacent articulating members (1a, 1b) is formed by a deformation step following the pushing into one another, wherein contacting connecting regions (4) pushed parallel over one another are deformed complementarily.

18. A method for producing a deflection mechanism (10) for aligning described above, further characterized in that at least one connecting portion (4) of each articulating member (1) is designed to be resilient, wherein the connecting portions (4) lock together by a resilient deflection when pushed into one another.

Although the present invention has been fully described above with reference to preferred exemplary embodiments, it is not limited thereto, but can be modified in a variety of ways.

LIST OF REFERENCE SIGNS

| | |
|---|---|
| 1 | articulating member |
| 2 | base body |
| 3 | through-opening |
| 4 | connecting portion |
| 5 | end portion |
| 6 | tab |
| 7 | longitudinal direction |
| 8 | radial direction |
| 9 | dome |
| 10 | deflection mechanism |
| 11 | molded part |
| 12 | opening |
| 13 | articulation axis |

1. An articulating member for or in a deflection mechanism at a distal end of a surgical instrument, comprising:
a base body having an axial through-opening along a longitudinal axis, so that a sub-portion of a working channel of the surgical instrument can be formed with the through-opening or can be passed through the through-opening; and
a connecting portion arranged on at least one axial end portion of the base body, wherein the connecting portion is designed for radial engagement with a connecting portion of a further articulating member, so that a positive and articulated connection can be formed between two articulating members.

2. The articulating member of claim 1, wherein the connecting portion is designed to be resilient, wherein the positive connection can be established by a resilient deflection in the radial direction.

3. The articulating member of claim 1, wherein the connecting portion has at least one tab.

4. The articulating member of claim 3, wherein the at least one tab projects beyond the base body in a longitudinal direction of the articulating member and is resiliently deflectable in a radial direction.

5. The articulating member of claim 3, wherein the at least one tab is dome-shaped, wherein the positive and articulated connection can be formed by the tab contacting a tab of a further articulating member, which tab is complementary to the dome-shaped tab.

6. The articulating member of claim 4, wherein the at least one tab is dome-shaped, wherein the positive and articulated connection can be formed by the tab contacting a tab of a further articulating member, which tab is complementary to the dome-shaped tab.

7. The articulating member of claim 5, wherein the dome-shaped tab has a convex or concave dome in a radial direction of the base body.

8. The articulating member of claim 1, wherein two tabs are provided on at least one end portion, which are formed opposite one another on a cross section of the end portion and/or mirrored to one another.

9. The articulating member of claim 1, wherein a connecting portion is arranged at each of two end portions which are arranged opposite one another on the base body, so that the articulating member can be connected to a further articulating member at each end portion.

10. The articulating member of claim 9, wherein the tab is designed for articulated engagement with a connecting portion of the further articulating member on a predetermined articulation axis.

11. The articulating member of claim 10, wherein the through-opening is designed for the passage and defined accommodation of steering means, in particular, steering cables or steering wires.

12. A deflection mechanism for aligning tools and/or for aligning a light, flushing and/or imaging unit, at a distal end of a surgical instrument, comprising:
a plurality of articulating members, each articulating member comprising a base body having an axial through-opening along a longitudinal axis, so that a sub-portion of a working channel of the surgical instrument can be formed with the through-opening or can be passed through the through-opening; and a connecting portion arranged on at least one axial end portion of the base body, wherein the connecting portion is designed for radial engagement with a connecting portion of a further articulating member, so that a positive and articulated connection can be formed between two articulating members, wherein
the articulating members are connected to one another in a positive and articulated manner such that an articulated working channel of the surgical instrument is formed through the through-openings or a flexible working channel can be passed through the through-opening.

13. The deflection mechanism of claim 12, wherein connecting portions of adjacent articulating members are arranged offset from one another in a plane, wherein, in a connected state, they are arranged one above the other and engage radially with one another.

14. The deflection mechanism of claim 13, wherein the connecting portions each have at least one tab, wherein at least one of the tabs is resiliently deflectable in a radial direction of the base body and in a connected state the tabs are locked together.

15. The deflection mechanism of claim 14, wherein the tabs of adjacent articulating members have a complementary shape.

16. The deflection mechanism of claim 15, wherein the complimentary shape is a complementary dome shape

17. The deflection mechanism of claim 12, wherein the deflection mechanism is configured for single use.

18. A method for producing a deflection mechanism for aligning tools and/or for aligning a light, flushing and/or imaging unit, at a distal end of a surgical instrument, comprising the steps of:
providing a plurality of articulating members, each articulating member comprising a base body having an axial through-opening along a longitudinal axis, so that a sub-portion of a working channel of the surgical instrument can be formed with the through-opening or can be passed through the through-opening; and a connecting portion arranged on at least one axial end portion of the base body, wherein the connecting portion is designed for radial engagement with a connecting portion of a further articulating member, so that a positive and articulated connection can be formed between two articulating members; and
pushing, at least partially, adjacent articulating members into one another, wherein connecting portions of adjacent articulating members are displaced relative to one another until the connecting portions contact one another in a radial direction of the base body such that a positive and articulated connection of the articulating members is produced or can be produced.

19. The method according to claim 18, wherein a connection of two adjacent articulating members is formed by a deformation step following the pushing into one another, wherein contacting connecting regions pushed parallel over one another are deformed complementarily.

20. The method according to claim 18, wherein at least one connecting portion of each articulating member is designed to be resilient, wherein the connecting portions lock together by a resilient deflection when pushed into one another.
